(19) United States
(12) Patent Application Publication
(10) Pub. No.: US 2010/0249038 A1
(43) Pub. Date: Sep. 30, 2010

(54) ANTAGONISTS OF THE RECEPTOR FOR ADVANCED GLYCATION END-PRODUCTS (RAGE)

(75) Inventors: Craig D. Logsdon, Houston, TX (US); Thiruvengadam Arumugam, Pearland, TX (US)

Correspondence Address: Nielsen IP Law LLC, 1177 West Loop South, Suite 1600, Houston, TX, 77027, US

(73) Assignee: Board of Regents, University of Texas System

(21) Appl. No.: 12/664,056

(22) PCT Filed: Jun. 12, 2008
(86) PCT No.: PCT/US08/66748

§ 371 Date: Dec. 10, 2009

Related U.S. Application Data
(60) Provisional application No. 60/943,468, filed on Jun. 12, 2007.

(51) Int. Cl. A61K 38/10; C07K 7/06; C07K 7/08; C07H 21/04; A61K 31/711; A61K 38/08; A61P 35/00

(52) U.S. Cl. 514/19.3; 530/328; 530/327; 536/23.5; 514/44 R

(57) ABSTRACT

Novel peptides are useful as antagonists of RAGE and may be used to treat cancer, inflammation, diabetes and arthritis through the administration of a therapeutically effective amount of the peptide to a subject in need thereof.

### CROSS-REFERENCE TO RELATED APPLICATIONS

This application claims priority to U.S. patent application Ser. No. 60/943,468 filed Jun. 12, 2007. This application is incorporated by reference herein it its entirety.

### FIELD OF THE INVENTION

Antagonists of Receptor for Advanced Glycation End-products (“RAGE”) to treat disease are disclosed.

### STATEMENT REGARDING FEDERALLY SPONSORED RESEARCH OR DEVELOPMENT

This work was supported by funds from the Lockton Endowment at M.D. Anderson Cancer Center.

### THE NAMES OF THE PARTIES TO A JOINT RESEARCH AGREEMENT

None.

### REFERENCE TO SEQUENCE LISTING

The Sequence Listing is contained on an electronic text file named Sequence_Listing.txt which is 3.21 KB in size and was created on Jun. 12, 2008. The material contained in the .txt file is being filed concurrently via USPTO EFS-Web with the present specification and is hereby incorporated-by-reference.

### BACKGROUND OF THE INVENTION

The receptor for advanced glycation end-products (“RAGE”) is a member of the immunoglobulin superfamily of receptors. RAGE is expressed in most tissues and is present on many different types of cells. Ramasamy, R., et al., (2005) Glycobiology 15, 16R-28R (‘Ramasamy’); Bierhaus, A., et al., (2005); J. Mol. Med. 83, 876-886 (‘Bierhaus 2005’); Bierhaus, A., et al., (2006) Curr. Opin. Investig. Drugs 7, 985-991 (‘Bierhaus 2006’). The RAGE gene is localized on chromosome 6 near the human leukocyte antigen locus of the MHC III complex in humans and mice, in close proximity to the homeobox gene HOX12 and the human counterpart of the mouse mammary tumor gene int-3. Sugaya, K., et al., (1994) Genomics 23, 408-419.

RAGE is expressed in a variety of human cancers, including ovarian, breast, colonic, brain, lung, prostate, lymphoma, and melanoma. Logsdon C D, Fuentes M K, Huang E H, Arumugam T., RAGE and RAGE Ligands in Cancer, (2007) Curr Mol. Med. Dec; 7(8):777-89. (Pubmed id: 18331236). Increased levels of RAGE have been reported in certain cancers, including prostate, colon, and gastric tumors. Ishiguro, H., et al., (2005) Prostate 64, 92-100; Sasahira, T., et al., (2005) Virchows Arch. 446, 411-415; Kuniyasu, H., et al., (2002) J. Pathol. 196, 163-170. On the other hand, in lung cancer, RAGE levels are significantly decreased. Bartling, B., et al., (2005) Carcinogenesis 26, 293-301; Hofmann, H. S., et al., (2004) Am. J. Respir. Crit. Care Med. 170, 516-519; Schraml, P., et al., (1997) Cancer Res. 57, 3669-3671; Fuentes, M. K., et al., RAGE Activation by S100P Stimulates Colon Cancer Cell Growth, Migration and Cell Signaling Pathways, Diseases of the Colon and Rectum (2007). But alterations in RAGE splice variants in lung cancer suggests that together with decrease in RAGE, there is also a decrease in a splice variant that acts as a natural antagonistic form of the receptor. Kobayashi, S., et al., (2007) Am. J. Respir. Crit. Care Med. 175, 184-189.

Noteworthy, RAGE levels are found to be elevated in arthritis, Alzheimer's disease and diabetes. Yan, S. F., et al., (2004) Diab. Vasc. Dis. Res. 1, 10-20; Tan, K. C., et al., (2006) Diabetologia 49, 2756-2762; Sunahori, K., et al., (2006) Arthritis Rheum. 54, 97-104; Sasaki, N., et al., (2001) Brain Res. 888, 256-262; See also, Ramasamy, R., et al., (2005) Glycobiology 15, 16R-28R; Bierhaus, A., et al., (2005) J. Mol. Med. 83, 876-886; and Bierhaus, A., et al., (2006) Curr. Opin. Investig. Drugs 7, 985-991. Furthermore, RAGE is reported as a mediator of vascular dysfunction in diabetes. Goldin, A., et al., (2006) Circulation 114, 597-605 (‘Goldin’); Yan, S. F., et al., (2004) Diab. Vasc. Dis. Res. 1, 10-20. Physiologically, RAGE reportedly has a role in embryonic neuronal outgrowth. Srikrishna, G., et al., (2002) J. Neurochem. 80, 998-1008. In the adult, RAGE appears to act primarily in pathological responses as a receptor for a very broad range of ligands that fall into the category of damage-associated molecular pattern molecules (DAMPs).

In a variety of models of inflammation and disease, expression of the RAGE ligands is also elevated. Indeed, RAGE ligands are over-expressed in many types of cancer. RAGE ligands such as high-mobility group box-1 (HMGB1 or HMG-1, also called amphoterin) and members of the S100/calgranulin family of proteins, are up-regulated in both cancer and inflammation. Inhibition of RAGE reduces the growth, motility, and invasiveness of natural and implanted tumors in nude mice. Taguchi, A., et al., (2000) Nature 405, 354-360.

### BRIEF SUMMARY OF THE INVENTION

Peptides comprising the amino acid of SEQ. ID. NO. 1, 2, 3, 4, or 5 are presented herein. The peptides disclosed herein are useful as antagonists of RAGE and may include N-terminal (acetylation, glycosylation) or C-terminal (amidation) modifications, the use of unnatural amino acids (e.g. beta-amino and α-trifluoromethyl amino acids) particularly at labile sites, cyclization and coupling with carriers such as polyethylene glycol (PEG). Methods of treating cancer, inflammation, diabetes and arthritis by the administration of a therapeutically effective amount of the peptide to a subject in need thereof are provided.

### BRIEF DESCRIPTION OF THE DRAWINGS

The foregoing summary as well as the following detailed description of the preferred embodiment of the invention will be better understood when read in conjunction with the appended drawings. It should be understood, however, that the invention is not limited to the precise arrangements and instrumentalities shown herein.

For a more complete understanding of the present invention, and the advantages thereof, reference is now made to the following descriptions taken in conjunction with the accompanying drawings, in which:

FIG. 1 depicts the molecule structure of RAGE and its splice variants.

FIG. 2 is a schematic of the various cancer and other cells in the tumor microenvironment that can interact with RAGE.

FIG. 3A and FIG. 3B show S100P expression increases and silencing reduces tumor growth in vivo. FIG. 3A depicts calculated tumor volume after 4 weeks after athymic mice were inoculated subcutaneously with 1×10⁶ of either vector transfected or S100P expressing Panc-1 cells. FIG. 3B depicts estimated tumor volume from six animals after six weeks by bioluminescent imaging and after being treated with BxPC3 cells stably transfected with control siRNA or S100P shRNA.

FIG. 4A and FIG. 4B show S100P stimulates HUVEC cell proliferation. Specifically, FIG. 4A depicts the increase of HUVEC cells after being culture in the presence of either S100P or uniinduced bacterial protein for 48 hours. FIG. 4B is the CD31 staining of tumor formed from pancreatic cancer cells orthotopically implanted in nude mice. FIG. 4C shows S100P stimulates endothelial cell interaction after 4 hours.

FIG. 5 depicts that S100P interacts directly with RAGE as RAGE was identified in the immunoprecipitates by western blotting with an anti-RAGE antibody (IB-RAGE).

FIGS. 6A and 6B show the effects of exogenous S100P are RAGE dependent. FIG. 6A shows cell proliferation of wild-type NIH3T3 plated at equal numbers and treated for 48 hours. FIG. 6B shows cell survival of cells treated with or without 5-FU.

FIG. 7A and FIG. 7B show that peptide antagonists can inhibit the binding of S100P to RAGE and S100P stimulation of pancreatic cancer cell NFκB activity.

FIG. 8A and FIG. 8B show in vivo inhibition by peptide antagonists on BxPC3 cell NFκB activity.

FIG. 9 depicts that peptide antagonists block binding of amphoterin (HMGB1 or HMG-1) to RAGE. (*=p<0.05 versus HMGB1 alone.)

FIG. 10 depicts that peptide antagonists block binding of S100P and S100A4 to RAGE. (*=p<0.05 versus S100P or S100A4 alone.)

FIG. 11 depicts that S100P induced NFκB activity is blocked by pretreatment of cells with peptide antagonists.

FIG. 12 depicts that peptide antagonists block NFκB activity in vivo in subcutaneous tumors.

FIG. 13 depicts that peptide antagonists block NFκB activity in vivo after intraperitoneal injection in orthotopic tumors.

FIG. 14 depicts inhibition of the binding of amphoterin to RAGE by antagonist peptides in vitro.

FIG. 15 depicts inhibition of the binding of S100P to RAGE by antagonist peptides in vitro.

FIG. 16 depicts inhibition of S100P activation of RAGE by antagonist peptides analyzed on pancreatic cancer cells in vitro.

### DETAILED DESCRIPTION OF THE INVENTION

Novel peptide antagonists of RAGE have been developed based on an examination of the structure of S100P. Known peptides are not suitable in vivo due to the large size of the peptide (30 mers plus) which causes an immune response and rapid clearance. The peptide antagonists disclosed herein are short, about 10 to 14 mers, and suitable for in vivo inhibition of RAGE.

The novel peptides have amino acid sequences are shown in Table 1.

TABLE 1

| SEQ ID NO | Amino Acid Sequence |
|---|---|
| 1 | ELKVLMEKEL |
| 2 | KELPGFLQSGKDKD |
| 3 | GKDGDAVDKLLKD |
| 4 | LEKEMLVKLE |
| 5 | DKLLKDVADGDKG |

Modifications can be made to the peptide antagonists in order to improve their stability and biological activity. Such modifications include N-terminal (acetylation, glycosylation) or C-terminal (amidation) modifications, the use of unnatural amino acids (e.g. beta-amino and α-trifluoromethyl amino acids) particularly at labile sites, cyclization and coupling with carriers such as polyethylene glycol (PEG). These peptides are designed to interfere with S100P activation of RAGE, to reduce tumor growth and metastasis, and increase the effectiveness of gemcitabine chemotherapy in preclinical animal models.

In the alternative or in addition, variants of the peptides disclosed herein may be produced. For example, conservative amino acid substitutions which retain the charge distribution structure of SEQ ID NOs: 1-5 and the ability of the modified peptide antagonist to bind RAGE may be made. A conservative substitution is one in which an amino acid is substituted for another amino acid that has similar properties, such that one skilled in the art of peptide chemistry would expect the secondary structure and hydropathic nature of the polypeptide to be substantially unchanged. Alanine is one example of an amino acid that may be substituted at any position within the peptide sequences disclosed herein where proper charge distribution would be retained.

A nucleic acid sequence which encodes SEQ ID NO:1 is provided as SEQ ID No:6 in Table 2 below.

TABLE 2

| SEQ ID NO | Nucleic Acid Sequence |
|---|---|
| 6 | GAGCTCAAGGTGCTGATGGAGAAGGAGCTA |

While it may be possible for the peptides to be administered as the raw chemical, it is also possible to present them as a pharmaceutical formulation. Accordingly, these peptides can be made part of a pharmaceutical formulation comprising a peptide together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a peptide or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration of the peptides may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active peptide doses.

The peptides may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the peptides may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such peptides may comprise the active ingredient in a flavored basis such as sucrose and acacia or tragacanth.

The peptides may also be formulated in rectal compositions such as suppositories or retention enemas, e.g., containing conventional suppository bases such as cocoa butter, polyethylene glycol, or other glycerides.

Peptides may be administered topically, that is by non-systemic administration. This includes the application of a peptide externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Preferred unit dosage formulations are those containing an effective dose, as herein below recited, or an appropriate fraction thereof, of the active ingredient.

It should be understood that in addition to the ingredients particularly mentioned above, the formulations may include other agents conventional in the art having regard to the type of formulation in question, for example those suitable for oral administration may include flavoring agents.

Preferred unit dosage formulations are those containing an effective dose, as herein below recited, or an appropriate fraction thereof, of the active ingredient.

It should be understood that in addition to the ingredients particularly mentioned above, the formulations may include other agents conventional in the art having regard to the type of formulation in question, for example those suitable for oral administration may include flavoring agents.

These peptides may be administered orally or via injection at a dose of from 0.1 to 500 mg/kg per day. The dose range for adult humans is generally from 5 mg to 2 g/day. Tablets or other forms of presentation provided in discrete units may conveniently contain an amount of peptide which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration. Hence, the phrase “therapeutically effective” is intended to qualify the amount of active ingredients used in the treatment of a disease or disorder. This amount will achieve the goal of reducing or eliminating the said disease or disorder. The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The peptides can be administered in various modes, e.g. orally, topically, or by injection. The precise amount of peptide administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

Alternatively, other viable and important options for peptide-based therapeutics involve introducing the peptide sequences presented herein as nucleic acids, either as direct DNA vaccines or recombinant vaccinia virus-based polyepitope vaccine. For example, DNA vaccines include naked and facilitated vaccines. Further, they may be administered by a variety of techniques that include several different devices and compositions for administering substances to tissue.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

Therapeutics which introduce peptide sequences as nucleic acids may be formulated into a vaccine in a neutral or salt form. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A), or emulsion with a 20% solution of a perfluorocarbon (Fluosol-DA®) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays.

In certain instances, it may be appropriate to administer at least one of the compounds described herein in combination with another therapeutic agent. By way of example only, if one of the side effects experienced by a patient upon receiving one of the peptides therein is hypertension, then it may be appropriate to administer an anti-hypertensive agent in combination with the initial therapeutic agent. Or, by way of example only, the therapeutic effectiveness of one of the peptides described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the peptides described herein with another therapeutic agent (which also includes a therapeutic regimen) that also has therapeutic benefit. By way of example only, in a treatment for diabetes involving administration of one of the peptides described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a novel peptide) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, methods for treating RAGE-mediated disorders in a human or animal subject in need of are presented. Such treatments include administering to a subject an amount of a peptide effective to reduce or prevent said disorder in the subject in combination with at least one additional agent for the treatment of said disorder that is known in the art. In a related aspect, therapeutic compositions comprising at least one peptide in combination with one or more additional agents for the treatment of RAGE-mediated disorders are provided.

The peptides can be used in the treatment of cancer including ovarian, breast, colonic, brain, lung, prostate, pancreatic, lymphoma and melanoma, arthritis, diabetes and inflammation and related disorders.

The basic structure of RAGE consists of three immunoglobulin-like regions, one “V”-type domain and two “C”-type domains, followed by a short transmembrane domain and a short cytoplasmic tail. Bierhaus, A., et al., (2005) J. Mol. Med. 83, 876-886; Schmidt, A. M., et al., (1994) J. Biol. Chem. 269, 9882-9888. Three RAGE isoforms are commonly referred to as the full-length RAGE receptor, expressed secretory RAGE (esRAGE), and N-truncated RAGE (NtRAGE). Schlueter, C., et al., (2003) Biochim. Biophys. Acta 1630, 1-6; Park, I. H., et al., (2004) Mol. Immunol. 40, 1203-1211. Before esRAGE was discovered as a naturally occurring, a synthetic version of this molecule, termed soluble RAGE (“sRAGE”), was produced in a baculovirus expression system as a means of inhibiting RAGE activation. Hofmann, M. A., et al., (1999) Cell 97, 889-901.

RAGE was originally discovered as the cell surface receptor for the advanced glycation end-products (AGEs), a heterogeneous population of protein and lipid adducts that are formed through a post-translational, non-enzymatic glycoxidation reaction of sugar ketones or aldehyde groups with free amino groups. Schmidt, A. M., et al., (1992) J. Biol. Chem. 267, 14987-14997; Dunn, J. A., et al., (1991) Biochemistry 30, 1205-1210; van Heijst, J. W., et al., (2005) Ann. N.Y. Acad. Sci. 1043, 725-733. However, RAGE is a member of the immunoglobulin superfamily, and based on the biology of this family, RAGE interacts with other ligands in addition to products of glycoxidation. Springer, T. A., (1990) Nature 346, 425-434.

As a receptor for a number of ligands, known ligands of RAGE include members of the 5100 family of proteins. Schmidt, A. M., et al., (2001) J. Clin. Invest 108, 949-955. S100 molecules are primarily known for their roles in inflammation. Hofmann, M. A., et al., (1999) Cell 97, 889-901. S100A4, S100B, and S100P have been shown to mediate cell functions via RAGE activation. Yammani, R. R., et al., (2006) Arthritis Rheum. 54, 2901-2911; Shaw, S. S., et al., (2003) Diabetes 52, 2381-2388; Arumugam, T., et al., (2004) J. Biol. Chem. 279, 5059-5065. Extracellular newly identified RAGE binding protein (EN-RAGE), now known as S100A12 is a molecule that bound to and activates RAGE in humans, but not in mice. Hofmann, M. A., et al., (1999) Cell 97, 889-901; Fuellen, G., et al., (2003) Trends Immunol. 24, 622-624. Other RAGE ligands include amyloid-β-peptide and β fibril sheets, involved in the development of Alzheimer's disease and HMGB1.

Despite its wide-spread distribution, under normal conditions, RAGE is inactive. Moreover, RAGE-deficient mice have been shown to develop and function essentially normally. Liliensiek, B., et al., (2004) J. Clin. Invest 113, 1641-1650. The increased expression of RAGE is likely to be a positive-feedback regulation of the RAGE promoter through RAGE activation of NFκB (also referred to herein as “NF-kappa B”) that has been shown to amplify and prolong inflammatory signals. Li, J., et al., (1997) J. Biol. Chem. 272, 16498-16506; Hofmann, M. A., et al., (1999) Cell 97, 889-901.

Although activation of RAGE has been shown to activate NF-kappa B, several studies have revealed that RAGE also influences other important intracellular signaling pathways, many of which are entirely independent of NF-kappa B. Examples include: (1) Advanced glycation end products, the ligands of RAGE, operating primarily through a caspase-8 activation of caspase-3 mechanism stimulate apoptosis in osteoblastic cells independently of effects on NF-kappa B, (Alikhani M., et al. (2007) Bone 40(2), 345-353); (2) RAGE activates Smad signaling to mediate diabetic complications in a manner independent of NF-kappa B (Li, J. H. (2004) FASEB J. 18, 176-178); (3) The RAGE mediated migration of immune cells, including dendritic cells, requires the autocrine/paracrine release of HMGB1 and the integrity of the RAGE pathway, without indication of NF-kappa B involvement (Dumitriu I. E. et al., (2007) J. Leukoc. Biol. 81, 84-91); (4) RAGE mediates neutrophil adhesion to and migration across intestinal epithelial monolayers in a process that appears to be mediated by CD11b/CD18 beta(2) integrin signaling that does not involve NF-kappa B (Zen K. et al, (2007) J. Immunol. 178, 2483-2490); (5) RAGE has also been shown to activate the vascular endothelial growth factor (VEGF) pathway via activation of hypoxia inducible factor-1 (HIF-1) in a process independent of NF-kappa B (Treins C. et al. (2001) J. Biol. Chem. 276, 43836-43841); (6) The janus kinase (JAK)/signal transducers and activators of transcription (STAT) cascade is also activated by RAGE in mesangial cells (Brizzi M. F. et al. (2004) FASEB J. 18, 1249-1251) and RAGE activation of this cascade was found to be responsible for AGE (advanced glycation end-product) induction of collagen production in NRK-49F cells (Huang J-S et al. (2001) J. Cell. Biochem. 81, 102-113); and (7) RAGE activation has been shown to induce cellular oxidant stress that triggers a cascade of intracellular signals involving p21(ras) and MAP kinase, culminating in activation of several transcription factors in addition to NF-kappa B. The molecular mechanism that triggers this pathway likely involves oxidant modification and activation of p21(ras) (Lander H. M. et al. (1997) J. Biol. Chem. 272, 17810-17814).

RAGE plays key roles in inflammation and angiogenesis, important elements of cancer progression. See Bierhaus, A., et al., (2006) Curr. Opin. Investig. Drugs 7, 985-991; Goldin, A., et al., (2006) Circulation 114, 597-605. Cancer cells as well as several other types of cells such as endothelial and smooth muscle cells, fibroblasts, and leukocytes, express RAGE. Activation of RAGE initiates a variety of cell signaling pathways that regulate important cellular functions, including proliferation, survival, migration, motility, and invasiveness. To fully understand the role of RAGE in cancer, differences in the levels of RAGE, its splice variants, and its ligands must be considered.

RAGE also mediates the autocrine effects of S100P to increase the proliferation, survival and invasiveness of cancer cells. RAGE is also expressed on tumor cells. RAGE contributes directly to the aggressive behavior of cancer cells by stimulating cell growth, resistance to therapy, invasiveness and metastatic potential. For example, in pancreatic cancer, RAGE is expressed and S100P is over-expressed in more than 94 percent of tumors. Activation of RAGE on endothelial cells increases endothelial permeability to macromolecules. RAGE also stimulates proliferation and tube formation of adult skin microvascular endothelial cell proliferation.

Blocking RAGE activation in vivo has been shown beneficial in a variety of diseases including cancer. Taguchi, A., et al., Blockade of RAGE-Amphoterin Signaling Suppresses Tumor Growth and Metastases, (2000) Nature 405, 354-360 (‘Taguchi’); Huttunen, H. J., et al., Receptor for Advanced Glycation End Products-Binding Cooh-Terminal Motif of Amphoterin Inhibits Invasive Migration and Metastasis,” (2002) Cancer Res. 62, 4805-4811. Indeed, RAGE activation has been inhibited experimentally in a variety of ways, including (1) expression of a dominant-negative truncated receptor; (2) treatment with sRAGE; (3) treatment with a blocking RAGE antibody; (4) treatment with an antagonistic peptide derived from HMGB1; (5) treatment with cromolyn to bind to RAGE ligand S100P; and (6) gene silencing with anti-sense oligonucleotides. Taguchi, A., et al., (2000) Nature 405, 354-360; Abe, R., et al., (2004) J. Invest. Dermatol. 122, 461-467 Huttunen, H. J., et al., (2002) Cancer Res. 62, 4805-4811; Arumugam, T., et al., Cromolyn Blocks S100P Activation of RAGE and Improves Gemcitabine Effectiveness in Pancreatic Cancer, J. Natl. Cancer Inst. 2006; Kuniyasu, H., et al., (2002) J. Pathol. 196, 163-170. These approaches are not likely to be clinically applicable.

The most widely used approach for investigating the role of RAGE has been sRAGE, a synthetic version of the naturally occurring secreted form of the receptor that can act to sequester RAGE ligands. sRAGE treatment also has been reported to be useful as a cancer treatment. Application of sRAGE was shown to suppress the growth of tumor cells in vitro and in vivo. Taguchi, A., et al., (2000) Nature 405, 354-360. However, sRAGE can influence disease processes, including diabetic nephropathy, neuropathy, and arterial restenosis, in RAGE-deficient animals, particularly, since RAGE ligands likely also interact with other receptors. Bierhaus, A., et al., (2005) J. Mol. Med. 83, 876-886. Therefore, administration of sRAGE may inhibit a variety of cellular pathways in addition to its effect on RAGE. Along these lines, sRAGE can directly affect cell function by interacting with cells expressing Mac-1, triggering a cellular response. Pullerits, R., et al., (2006) Arthritis Rheum. 54, 3898-3907. Also, sRAGE has been found to be chemotactic for leukocytes and to lead to their activation. Further, RAGE can interact with Mac-1 during cell-to-cell interactions. Chavakis, T., et al., (2003) J. Exp. Med. 198, 1507-1515. Moreover, sRAGE is a large molecule that is relatively difficult to produce and may provoke an undesirable immune response. Therefore, for several reasons, sRAGE is an experimental tool rather than a therapeutic treatment in the clinical setting.

In addition, it was recently demonstrated that the small molecule cromolyn, which is widely used to treat allergic symptoms, can bind the RAGE ligand S100P and prevent its activation of RAGE. Arumugam, T., et al., (2006) J. Natl. Cancer Inst. 98, 1806-1818. Cromolyn also binds to other S100 molecules, but it is unknown whether it will inhibit RAGE activation by these or other RAGE ligands. Oyama, Y., et al., (1997) Biochem. Biophys. Res. Commun. 240, 341-347. Cromolyn was found to inhibit pancreatic cancer cell function and pancreatic tumor formation in animal models, likely through its ability to block an autocrine loop involving S100P and RAGE. Arumugam, T., et al., (2006) J. Natl. Cancer Inst. 98, 1806-1818. Cromolyn has the advantage of having been used in humans for many years. However, cromolyn has other targets and it has some pharmacokinetic properties, including low oral bioavailability, that are not desirable. Shapiro, G. G., et al., (1985) Pharmacotherapy 5, 156-170. Currently, no small molecule inhibitors that target RAGE directly have been identified.

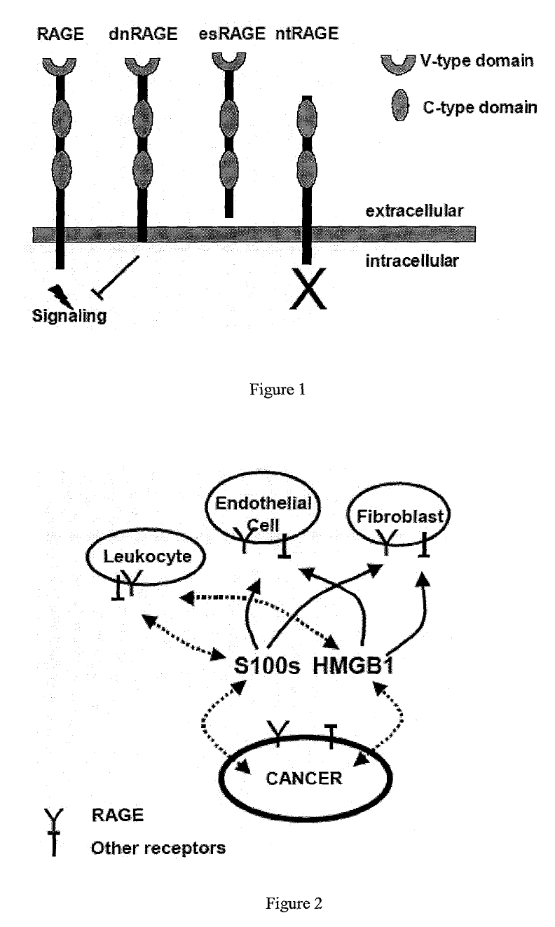

FIG. 1 depicts the molecular structure of RAGE and its splice variants. Full-length RAGE possesses one V-type and two C-type immunoglobulin domains. Dominant-negative RAGE (dnRAGE) has lost the cytoplasmic domain responsible for RAGE ligand-mediated signaling and can interfere with the signaling of the full-length receptor. Expressed secretory RAGE (esRAGE) lacks both the cytoplasmic domain and the transmembrane domain. This form of the receptor is secreted and can act as an antagonist by binding RAGE ligands. N-truncated RAGE (ntRAGE) lacks the V-type domain and therefore cannot bind RAGE ligands. However, all forms of RAGE are thought to be able to interact with Mac-1 on other cells.

The expression of the splice variant esRAGE has been investigated in several normal organs and was found to be present in a variety of cell types. Cheng, C., et al., (2005) Mod. Pathol. 18, 1385-1396. The mRNA for the esRAGE contains the same immunoglobulin domains present in the mRNA for the full-length RAGE receptor and also contains part of intron 9, which incorporates a stop codon within the sequence (FIG. 1). Because of the insertion of the stop codon, the esRAGE mRNA lacks exons 10 and 11, which encode the transmembrane domain of RAGE, resulting in esRAGE not being embedded in the membrane. Rather, esRAGE is efficiently secreted from cultured cells and is capable of capturing ligands. Yonekura, H., et al., (2003) Biochem. J. 370, 1097-1109. For this reason, esRAGE can function as a decoy-type receptor molecule.

Serum levels of expressed secretory RAGE (“esRAGE”) are altered under various disease states. Serum esRAGE levels are significantly higher in patients with type 2 diabetes and are positively associated with the presence of coronary artery disease and nephropathy. Nakamura, K., et al., (2006) Diabetes Metab Res. Rev; Tan, K. C., et al., (2006) Diabetologia 49, 2756-2762. Recently, levels of circulating esRAGE are found to be greatly reduced or absent in 75% of non-small cell lung cancers (NSCLCs). Kobayashi, S., et al., (2007) Am. J. Respir. Crit. Care Med. 175, 184-189. Hence, esRAGE may modify the activity of RAGE signaling. Recently, RAGE splice variants have been detected and appear to be numerous under pathological conditions.

There are also variant RAGE isoforms from the same gene (co-expressed with the full-length RAGE transcript) and the pre-mRNA of RAGE may be subjected to alternative splicing. Schlueter, C., et al., (2003) Biochim. Biophys. Acta 1630, 1-6. Spliced variants of RAGE have been found in several cell types, including endothelial cells and pericytes, brain astrocytes and peripheral blood mononuclear cells, and lung cells. Yonekura, H., et al., (2003) Biochem. J. 370, 1097-1109; Park, I. H., et al., (2004) Mol. Immunol. 40, 1203-1211; Kobayashi, S., et al., (2007) Am. J. Respir. Crit. Care Med. 175, 184-189. In pancreatic cancer cells, RAGE splice variants are expressed that are not expressed in the normal pancreas.

The mRNA for NtRAGE retains intron 1, which like intron 9 contains a novel stop codon, resulting in the loss of both exon 1 and exon 2. This truncated version of full-length RAGE therefore lacks the V-type immunoglobulin domain but is otherwise identical to full-length RAGE and is retained in the plasma membrane (FIG. 1). As a result of the deletion of the V-type immunoglobulin domain, NtRAGE is significantly impaired in its ability to bind RAGE ligands. Park, I. H., et al., (2004) Mol. Immunol. 40, 1203-1211; Yonekura, H., et al., (2003) Biochem. J. 370, 1097-1109; Ding, Q., et al., (2005) Neurosci. Lett. 373, 67-72. NtRAGE can interact with other molecules and interfere with normal functions that may be independent of signaling by the typical RAGE ligands. For example, expression studies with a plasmid bearing the N-truncated cDNA indicate that it expressed 42 kDa protein without N-linked oligosaccharides, which was localized mainly on the plasma membrane similar to full-length RAGE; but it is unclear how it reaches the plasma membrane, as this variant lacks a signal peptide. Yonekura, H., et al., (2003) Biochem. J. 370, 1097-1109. Expression of this non-binding variant did not inhibit AGE-stimulated effects. Nevertheless, overexpression of NtRAGE inhibited endothelial cell migration. Yonekura, H., et al., (2003) Biochem. J. 370, 1097-1109.

S100 molecules are small, calcium-binding, cell-signaling molecules of the EF-hand (helix-loop-helix) type. Marenholz, I., et al., (2004) Biochem. Biophys. Res. Commun. 322, 1111-1122; Foell, D., et al., (2007) J. Leukoc. Biol. 81, 28-37. These molecules can interact to form dimers or various oligomeric structures and have both intracellular and extracellular functions. As intracellular molecules, they are calcium-signaling or calcium-buffering proteins responsible for assorted roles in the cell cycle, cell differentiation, and cell motility. However, several S100 family members, including S100B, S100A4, S100A8, S100A9, S100A12, S100A13, and S100P, are secreted and appear to have extracellular roles. Secreted S100s have been long observed to collect at sites of chronic inflammation.

Extracellular newly identified RAGE binding protein (EN-RAGE), now known as S100A12 is a molecule that bound to and activates RAGE. Hofmann, M. A., et al., (1999) Cell 97, 889-901; Fuellen, G., et al., (2003) Trends Immunol. 24, 622-624. Similarly, S100A4, S100B, and S100P have been shown to mediate cell functions via RAGE activation. Yammani, R. R., et al., (2006) Arthritis Rheum. 54, 2901-2911; Shaw, S. S., et al., (2003) Diabetes 52, 2381-2388; Arumugam, T., et al., (2004) J. Biol. Chem. 279, 5059-5065. On the other hand, certain studies have suggested that S100A4, S100B, and the complex of S100A8/9 exert important extracellular effects that are independent of RAGE. Kiryushko, D., et al., (2006) Mol. Cell. Biol. 26, 3625-3638; Sorci, G., et al., (2003) Mol. Cell. Biol. 23, 4870-4881; Robinson, M. J., et al., (2002) J. Biol. Chem. 277, 3658-3665. Importantly, S100 proteins, like other RAGE ligands, have separate intracellular roles as well as separate receptors in addition to RAGE.

Of the twenty plus members of the S100 family, currently only a few have been implicated in cancer. Marenholz, I., et al., (2004) Biochem. Biophys. Res. Commun. 322, 1111-1122. For example, the S100 family member S100P, which was named after identification in the placenta, has been shown to directly interact with RAGE and to have an important role in cancer. Becker, T., et al., (1992) Eur. J. Biochem. 207, 541-547; Arumugam, T., et al., (2004) J. Biol. Chem. 279, 5059-5065; Arumugam, T., et al., (2005) Clin. Cancer Res. 11, 5356-5364; Arumugam, T., et al., (2006) J. Natl. Cancer Inst. 98, 1806-1818. In animal models, the overexpression or silencing of S100P in cancer cells forming orthotopic tumors was directly correlated with increased or decreased pancreatic cancer tumor growth, respectively. Arumugam, T., et al., (2005) Clin. Cancer Res. 11, 5356-5364; Arumugam, T., et al., (2006) J. Natl. Cancer Inst. 98, 1806-1818. Transfection of S100P into a benign, non-metastatic rat mammary cell line caused an increase in local muscle invasion and metastasis in a mouse model. Wang, G., et al., (2006) Cancer Res. 66, 1199-1207. Exogenous treatment of pancreatic cancer cell lines with S100P has been shown to stimulate cell proliferation, survival, migration, and invasion and activated the MAP kinase (Erk 1/2) and NFκB pathways. Arumugam, T., et al., (2004) J. Biol. Chem. 279, 5059-5065; Arumugam, T., et al., (2005) Clin. Cancer Res. 11, 5356-5364; Arumugam, T., et al., (2006) J. Natl.

Cancer Inst. 98, 1806-1818. The effects of treatment with extracellular S100P have been shown to be mediated by its interaction with RAGE, since blockade of this interaction prevented the effects of exogenous S100P on cell functions. Arumugam, T., et al., (2004) J. Biol. Chem. 279, 5059-5065. S100A4 is often considered a metastasis-inducing molecule, and this function has been recently reviewed. Garrett, S. C., et al., (2006) J. Biol. Chem. 281, 677-680.

S100B is also a potential cancer biomarker, as it is highly expressed in melanoma. Harpio, R. et al., (2004) Clin. Biochem. 37, 512-518. However, not all S100s appear to promote cancer. S100A2 is often found to be inversely related to S100A4, and an anti-tumorigenic, mechanistic role for S100A2 has been described in squamous cell carcinoma. Matsubara, D., et al., (2005) Cancer Sci. 96, 844-857; Tsai, W. C., et al., (2006) Mol. Cancer. Res. 4, 539-547.

Further, RAGE has an ability to bind and be activated by AGEs. AGEs are known to accumulate in a variety of circumstances, including during the aging process, in the presence of hyperglycemia such as occurs during diabetes, and during the course of inflammatory diseases, including renal failure. Dunn, J. A., et al., (1991) Biochemistry 30, 1205-1210; Goldin, A., et al., (2006) Circulation 114, 597-605; Hofmann, M. A., et al., (1999) Cell 97, 889-901. The most common AGE found in vivo is the N^{ε}-carboxymethylysine (CML), which results from the glycation of a lysine residue. Reddy, S., et al., (1995) Biochemistry 34, 10872-10878.

AGEs (the first known ligand of RAGE) is involved in the vascular complications of diabetes. Furthermore, the presence or role of AGEs in cancer has been reported in at least one study where specific antibodies were used to localize CML and other AGEs in a variety of tumors. van Heijst, J. W., et al., (2005) Ann. NY. Acad. Sci. 1043, 725-733. Tumors are generally characterized by increased glucose uptake and a high rate of glycolysis, so the formation of AGEs might be expected. Also in another study, treatment with AGEs induced melanoma cell proliferation, migration, and invasion in vitro, and these effects were completely blocked by treatment with an antibody against RAGE. Abe, R., et al., (2004) J. Invest. Dermatol. 122, 461-467.

AGEs are involved in the growth and invasion of melanoma through interactions with RAGE. However, several other RAGE ligands are also likely to be expressed and secreted by tumor cells. Therefore, the specific contribution of AGEs is currently unknown. Further, AGEs may not stimulate cellular responses via RAGE, but, rather, contaminants in the preparation of AGEs might contribute to the apparent activation of RAGE. Valencia, J. V., et al., (2004) Diabetologia 47, 844-852. However, this study conflicts with a wealth of data showing AGE activation of RAGE.

RAGE was the first known receptor for HMGB1, as the two molecules were observed to be co-localized in the developing rat brain and their interaction was found to mediate neurite outgrowth. Hori, O., et al., (1995) J. Biol. Chem. 270, 25752-25761. Several studies have shown, via inhibition of RAGE signaling by approaches including treatment with soluble RAGE and expression of dominant-negative RAGE or blocking antibodies, that HMGB1 acts via RAGE. Taguchi, A., et al., (2000) Nature 405, 354-360.

Blocking the signaling cascade between HMGB1 and RAGE decreased tumor growth and metastasis in glioma cells. Taguchi, A., et al., (2000) Nature 405, 354-360. In this study, rat C6 glioma cells were stably transfected with RAGE mutated constructs and injected into nude mice. In vivo, tumor growth and metastasis were markedly decreased. In vitro, it was shown that blocking the HMGB1-RAGE interaction decreased cell proliferation, migration, and invasion. More recently, HMGB1 has been shown to activate the Toll-like receptor (TLR) pathways, specifically TLR2 and TLR4. Park, J. S., et al., (2006) Am. J. Physiol Cell Physiol 290, C917-C924; Yu, M., et al., (2006) Shock 26, 174-179. However, this possibility remains controversial, as one report suggests that bacterial endotoxins may be contaminants in preparations of HMGB1 and that an HMGB1 preparation that is endotoxin-free does not stimulate TLR. Rouhiainen, A., et al., (2007) J. Leukoc. Biol. 81, 49-58. Because HMGB1 has several effects, acts both intracellularly and extracellularly, and can potentially interact with more than one receptor, caution must be used when attempting to understand the role of RAGE in the actions of this molecule in disease.

HMGB1 was first referred to as amphoterin, because of its dipolar nature, and was thought to be a non-histone-binding protein because it was originally discovered to be bound loosely to chromatin. Lotze, M. T., et al., (2005) Nat. Rev. Immunol. 5, 331-342. Since that time, HMGB1 has been implicated in a variety of biologically important processes, including transcription, DNA repair, differentiation, neural development, and extracellular signaling, and its potential roles in cancer have recently been reviewed. As a nuclear protein, HMGB1 binds to the minor groove of DNA and facilitates the assembly of site-specific DNA binding proteins like p53 at their cognate binding sites within chromatin. Thomas, J. O., (2001) Biochem. Soc. Trans. 29, 395-401.

It is now known that HMGB1 also has an important extracellular function. HMGB1 can be secreted from activated inflammatory cells (e.g., monocytes and macrophages) or released from necrotic but not apoptotic cells and act as an extracellular cytokine. Wang, H., et al., (1999) Science 285, 248-251; Scaffidi, P., et al., (2002) Nature 418, 191-195; Lotze, M. T., et al., (2005) Nat. Rev. Immunol. 5, 331-342. When released from damaged cells, this molecule has been found to act as a “necrotic marker” used by the immune system to recognize tissue damage, initiate reparative responses, and promote maturation of lymphocytes. Ulloa, L., et al., (2006) Cytokine & Growth Factor Reviews 17, 189-201. Extracellular HMGB1 further acts as a potent pro-inflammatory cytokine, contributing to the pathogenesis of a wide variety of inflammatory disorders.

HMGB1 has several effects that increase the aggressiveness of cancer. One of its major effects is the stimulation of metastasis through its effects on the transcription of many genes involved at different steps in the metastatic cascade. Evans, A., et al., (2004) J. Surg. Oncol. 88, 86-99. HMGB1 also affects cancer cell survival; overexpression of HMGB1 was associated with reduced levels of pro-apoptotic genes and increased levels of anti-apoptotic genes. Volp, K., et al., (2006) Gut 55, 234-242; Brezniceanu, M. L., et al., (2003) FASEB J. 17, 1295-1297.

However, there is a conflicting report in which HMGB1 seems to have a pro-apoptotic effect on some cells. Kuniyasu, H., et al., (2005) Am. J. Pathol. 166, 751-760. A growing number of studies support the idea that HMGB1 is a useful therapeutic target in cancer and a number of other important diseases, including sepsis, acute respiratory distress syndrome, and arthritis. Taguchi, A., et al., (2000) Nature 405, 354-360; Lotze, M. T., et al., (2005) Nat. Rev. Immunol. 5, 331-342; Huttunen, H. J., et al., (2002) Cancer Res. 62, 4805-4811.

As depicted in FIG. 2, cancer cells and cells in the tumor microenvironment, including leukocytes, endothelial cells, and fibroblasts, express RAGE. RAGE ligands secreted from cancer cells or leukocytes can interact with RAGE and other mechanisms to influence tumor progression.

The role of RAGE in lung tumors has recently been reviewed. Franklin, W. A., (2007) Am. J. Respir. Crit. Care Med. 175, 106-107. Lung cancer is unique in that there is conflicting evidence concerning RAGE and RAGE ligands in this disease. Reduced levels of RAGE have been observed in NSCLC compared with the normal lung. Bartling, B., et al., (2005) Carcinogenesis 26, 293-301; Hofmann, H. S., et al., (2004) Am. J. Respir. Crit. Care Med. 170, 516-519; Schraml, P., et al., (1997) Cancer Res. 57, 3669-3671. Down-regulation of RAGE also correlated with higher tumor stages. Bartling, B., et al., (2005) Carcinogenesis 26, 293-301. Furthermore, overexpression of full-length human RAGE in lung cancer cells (NCI-H358) resulted in diminished tumor growth compared to that in dominant-negative RAGE-expressing cells in vivo. Bartling, B., et al., (2006) Am. J. Respir. Cell Mol. Biol. 34, 83-91. Recently, it was found that esRAGE, the splice variant that is secreted and acts as an antagonist, is also down-regulated in NSCLC. Kobayashi, S., et al., (2007) Am. J. Respir. Crit. Care Med. 175, 184-189. The balance of full-length RAGE and esRAGE may influence the ability of the cells to respond to endogenous ligands and is an example of the complexities of the role of RAGE in cancer. Furthermore, several RAGE ligands are highly expressed in the lung, including S100A12 and HMGB1. Li, J., et al., (1997) J. Biol. Chem. 272, 16498-16506. These ligand levels are critical determinants of RAGE function.

Other ligands are also expressed in lung cancer. The RAGE ligand S100P is overexpressed in NSCLC and associated with poor survival. Beer, D. G., et al., (2002) Nat. Med. 8, 816-824; Diederichs, S., et al., (2004) Cancer Res. 64, 5564-5569. Furthermore, forced expression of S100P in an NSCLC cell line increased its transendothelial migration. Diederichs, S., et al., (2004) Cancer Res. 64, 5564-5569. S100A4 is also up-regulated in NSCLC tissue and associated with poor patient survival. Kimura, K., et al., (2000) Int. J. Oncol. 16, 1125-1131. A recent study has shown that reduced E-cadherin expression combined with higher S100A4 expression is associated with poor prognosis due to increased metastasis in pulmonary adenocarcinoma. Miyazaki, N., et al., (2006) Int. J. Oncol. 28, 1369-1374.

Breast cancer has been a source of considerable information about RAGE ligands. In particular, S100A4 plays a crucial role in breast cancer growth. A direct correlation has been observed between S100A4 expression and mean vessel density in breast tumors. Hsieh, H. L., et al., (2003) Biochem. Biophys. Res. Commun. 307, 375-381. In one study, the long-term survival rate was much higher in S100A4-negative patients compared to S100A4-positive patients (80% vs. 11%, median follow-up 19 years). Rudland, P. S., et al., (2000) Cancer Res. 60, 1595-1603. S100A4 has also been shown to be an independent predictor of patient survival and a marker for early metastasis. Lee, W. Y., et al., (2004) Oncology 66, 429-438. S100P also plays an important role in breast cancer progression from initial tumorigenesis to invasive carcinoma.

S100P is specifically expressed in breast cancer tissue. Carlsson, H., et al., (2005) Int. 0.1 Oncol. 27, 1473-1481. Immunohistochemical analysis of S100P in 303 breast cancer patients followed up for up to 20 years has shown that the survival duration of patients with S100P-positive carcinomas was significantly worse—by about 7-fold—than that for those with S100P-negative staining. Wang, G., et al., (2006) Cancer Res. 66, 1199-1207. Moreover, patients with tumors that stained positively for both S100P and S100A4 had significantly shorter survival compared to patients with tumors positive for either S100 protein alone. Hence, the combination of S100P and S100A4 is the most significant independent risk factor for death in this group of patients. Wang, G., et al., (2006) Cancer Res. 66, 1199-1207. S100P seems to be increased early in tumorigenesis, as it is expressed in non-transformed breast epithelial cell lines after immortalization and also in hyperplastic ductal tissues. Guerreiro, D. S., et al., (2000) Int. J. Oncol. 16, 231-240.

Expression of another RAGE ligand, S100A9, was associated with poor differentiation in breast cancer tissues. Arai, K., et al., (2004) Eur. J. Cancer 40, 1179-1187. High levels of HMGB1 have also been observed in human primary breast carcinoma, and this expression was further enhanced by estrogen. Flohr, A. M., et al., (2001) Anticancer Res. 21, 3881-3885; Brezniceanu, M. L., et al., (2003) FASEB J. 17, 1295-1297; Lum, H. K. et al., (2001) Biochim. Biophys. Acta 1520, 79-84.

While changes in RAGE expression have not been reported in prostate cancer, RAGE ligands are involved in tumor initiation and metastasis. Ishiguro, H., et al., (2005) Prostate 64, 92-100; Hermani, A., et al., (2006) Exp. Cell Res. 312, 184-197; Kuniyasu, H., et al., et al., (2003) Oncol. Rep. 10, 1863-1868. The RAGE ligands S100A8 and S100A9 are overexpressed in human prostate cancer, and these proteins were co-localized with RAGE in cancer cells and secreted by prostate cancer cells. Hermani, A., et al., (2006) Exp. Cell Res. 312, 184-197.

The presence of the S100s, induced activation of NFκB, phosphorylation of p38, and MAP kinase (Erk1/2) activity and increased the migration of benign prostate cells in vitro. Hermani, A., et al., (2006) Exp. Cell Res. 312, 184-197. S100A9 serum levels were also found to be significantly elevated in patients with prostate cancer compared with those with benign prostatic hypertrophy or healthy individuals, and it was suggested that S100A9 could be a serum marker like prostate-specific antigen. Hermani, A., et al., (2005) Clin. Cancer Res. 11, 5146-5152. HMGB1 also appears to be involved in prostate cancer development. Ishiguro, H., et al., (2005) Prostate 64, 92-100. High levels of RAGE and HMGB1 have been observed in untreated prostate cancer tissue, hormone-refractory prostate cancer tissue, and a hormone-independent prostate cancer cell line compared to levels in normal prostate tissue. Ishiguro, H., et al., (2005) Prostate 64, 92-100. HMGB1-RAGE expression was also found to be elevated in PC-3 cells, and in these cells, androgen deprivation increased HMGB1 secretion and cancer cell invasion. Kuniyasu, H., et al., et al., (2003) Oncol. Rep. 10, 1863-1868.

In colon cancer, RAGE expression has been reported to increase as colon cancer progresses, as indicated by Dukes' classifications. See e.g., Kuniyasu, H., et al., (2003) Oncol. Rep. 10, 445-448; But see, Fuentes, M. K., et al., RAGE Activation by S100P Stimulates Colon Cancer Cell Growth, Migration and Cell Signaling Pathways, Diseases of the Colon and Rectum (2007). Immunohistochemical studies indicate that RAGE has three patterns of staining in colon cells: cytosolic, luminal, and membranous. A relationship between the RAGE staining pattern and atypia has also been reported; as atypia becomes more severe, RAGE localization moves from the cytosol to the membrane, suggesting that RAGE could be used for predicting malignant potential. Kuniyasu, H., et al., (2003) Oncol. Rep. 10, 445-448.

RAGE is also involved in the interface between inflammation and carcinogenesis in the colon. The multiple intestinal neoplasia (MIN+/−) mouse is the murine corollate of the human condition familial adenomatous polyposis (FAP). The phenotype of this model typically includes 20-50 adenomatous polyps, predominantly in the small bowel. It has been found that administration of sRAGE intraperitoneally from weaning to 20 weeks of age led to a significant decrease in the number of polyps. RAGE has also been documented to play a role in the inflammatory neoplastic model of the IL-10 null mouse. In this transgenic model, the incidence of chronic inflammatory enterocolitis is as high as 60% in some environments, with an incidence of dysplastic lesions of up to 30%. Berg, D. J., et al., (1996) J. Clin. Invest 98, 1010-1020. Furthermore, breeding the MIN+/−mouse with the IL-10 null mouse caused a dramatic increase in both inflammation and colonic polyps, supporting a role for inflammation in neoplasia. Huang, E. H., et al., (2006) Surgery 139, 782-788. The administration of sRAGE was able to ameliorate the inflammation observed in this model. Hofmann, M. A., et al., (1999) Cell 97, 889-901. Therefore, RAGE antagonism, by ameliorating inflammation, can be used in cancer prevention as well as in cancer therapy.

S100P is reportedly involved in the inflammation-to-carcinogenesis progression that occurs in colon cancer. S100P is elevated in the chronic inflammatory conditions of ulcerative colitis and Crohn's disease, which both increase the risk of colon cancer up to 10-fold. Ekbom, A., et al., (1990) N. Engl. J. Med. 323, 1228-1233. S100P was also overexpressed in flat adenomas of the colon, which are associated with a higher potential for malignancy compared to other adenomas. Kita, H., et al., (2006) J. Gastroenterol. 41, 1053-1063. S100P was found to be overexpressed in colon cancer tissue compared to matched normal counterparts. Fuentes, M. K., et al., RAGE Activation by S100P Stimulates Colon Cancer Cell Growth, Migration and Cell Signaling Pathways, Diseases of the Colon and Rectum (2007). In that study, S100P treatment increased proliferation and migration and activated the MAP kinase pathway (Erk1/2) and NFκB in SW480 colon cancer cells, and inhibiting the S100P/RAGE interaction blocked these biological effects. Fuentes, M. K., et al., RAGE Activation by S100P Stimulates Colon Cancer Cell Growth, Migration and Cell Signaling Pathways, Diseases of the Colon and Rectum (2007). As in pancreatic cancer, silencing S100P decreased tumor growth in a xenograft model of colon cancer. Doxorubicin-resistant colon cancer cell lines expressed higher levels of S100P when compared with their sensitive counterparts. Bertram, J., et al., (1998) Anticancer Drugs 9, 311-317. S100A4 is also expressed in colon cancer and is associated with invasive potential, as it has been found to be specifically overexpressed in invasive carcinoma rather than adenoma or normal tissue. Takenaga, K., et al., (1997) Clin. Cancer Res. 3, 2309-2316; Taylor, S., (2002) Br. J. Cancer 86, 409-416. In another study, S100A4 levels correlated with colon cancer patient survival. Gongoll, S., et al., (2002) Gastroenterology 123, 1478-1484. S100A4 overexpression in colon cancer samples has also been associated with gene hypomethylation. Nakamura, N., et al., (1998) Clin. Exp. Metastasis 16, 471-479. AGEs were shown to stimulate MAP kinase (Erk1/2) activation in one colon cancer cell line. Zill, H., et al., (2001) Biochem. Biophys. Res. Commun. 288, 1108-1111.

HMGB1 has been associated with invasion and metastasis of colon cancer, and it has also been studied in colon cancer cell lines in vitro. Kuniyasu, H., et al., (2003) Oncol. Rep. 10, 445-448. Colon cancer cell lines with reduced endogenous HMGB1 levels had decreased growth, migration, invasion, and activation of various cell signaling pathways, and these effects were reversed when cells were treated with conditioned medium containing HMGB1. Kuniyasu, H., et al., (2003) Int. J. Cancer 104, 722-727. Immunohistochemical studies have also linked RAGE with its ligand HMGB1 in colon cancer progression, as co-expression of RAGE and HMGB1 is closely associated with the invasion and metastasis of colorectal cancer. Kuniyasu, H., et al., (2003) Oncol. Rep. 10, 445-448.

There is no direct evidence showing that RAGE is overexpressed in pancreatic tumors, as neither quantitative reverse transcription polymerase chain reaction nor western blotting indicated differences between pancreatic tumor samples and normal controls. (unpublished observation). However, RAGE is expressed by pancreatic cancer cells, and the expression levels of RAGE in pancreatic cancer cell lines were reported to correspond with metastatic potential. Takada, M., et al., (2001) Hepatogastroenterology 48, 1577-1578. Also, RAGE exists as multiple splice variants in pancreatic cancer but as a single, full-length mRNA in the normal pancreas. (unpublished observation).

In contrast to RAGE itself, RAGE ligands are overexpressed in pancreatic cancer, as revealed by microarray and tissue array analysis of pancreatic cancer tissues. Logsdon, C. D., et al., (2003) Cancer Res. 63, 2649-2657; Ohuchida, K., et al., (2006) Clin. Cancer Res. 12, 5417-5422; Ohuchida, K., et al., (2005) Clin. Cancer Res. 11, 7785-7793; Crnogorac-Jurcevic, et al., (2003) J. Pathol. 201, 63-74. In particular, the molecule S100P has been found to be overexpressed in pancreatic cancer. Logsdon, C. D., et al., (2003) Cancer Res. 63, 2649-2657; Crnogorac-Jurcevic, et al., (2003) J. Pathol. 201, 63-74; Ohuchida, K., et al., (2006) Clin. Cancer Res. 12, 5411-5416. This expression is specific to pancreatic cancer and was not observed in samples of chronic pancreatitis, an inflammatory disease with similar histological features. Logsdon, C. D., et al., (2003) Cancer Res. 63, 2649-2657.

As was observed in breast cancer, S100P seems to be an early marker of premalignancy, as S100P expression has been shown to increase during pancreatic cancer progression from precursor PanIN lesions to invasive adenocarcinoma. Dowen, S. E., et al., (2005) Am. J. Pathol. 166, 81-92. The overexpression of S100P in pancreatic cancer has been suggested to be due to hypomethylation of its gene in pancreatic cancer. Sato, N., et al., (2004) Oncogene 23, 1531-1538. S100P was found to be secreted from pancreatic cancer cell lines and to act extracellularly through RAGE. Arumugam, T., et al., (2005) Clin. Cancer Res. 11, 5356-5364. Moreover, expression of S100P increased pancreatic orthotopic tumor growth and metastasis in vivo, and silencing of S100P had the opposite result. S100A4 has also been found to be involved in pancreatic cancer. S100A4 expression correlated significantly with higher pathological stage and poorer prognosis in an immunohistochemical analysis of tumor samples, and combining the analysis of S100A4 with that of E-cadherin improved the prognostic value of each marker. Oida, Y., et al., (2006) Oncol. Rep. 16, 457-463. Similar to S100P, overexpression of S100A4 in pancreatic cancer was related to gene methylation status. Sato, N., et al., (2003) Cancer Res. 63, 4158-4166.

In a variety of other cancers there is evidence that RAGE and/or RAGE ligands may be important. For example, RAGE expression appears to be closely associated with the invasiveness of oral squamous cell carcinoma, as silencing RAGE protein expression using an anti-sense oligomer reduced cancer cell migration and invasion of oral carcinoma cells in an animal model. Bhawal, U. K., et al., (2005) Oncology 69, 246-255. S100P has also been identified as a gene highly expressed in oral squamous cell carcinoma. Kupferman, M. E., (2006) Oral Oncol. In melanoma, RAGE was detected in the cytoplasm of human melanoma cells (G361 and A375), and these cells were stimulated to proliferate and migrate after treatment with AGEs. Abe, R., et al., (2004) J. Invest. Dermatol. 122, 461-467. Furthermore, anti-RAGE antibody inhibited tumor formation, reduced invasion, and increased survival in an animal model in vivo. Abe, R., et al., (2004) J. Invest. Dermatol. 122, 461-467.

In melanoma, it has also been reported that AGEs were present in the beds of human melanoma tumors, whereas they were nearly undetectable in normal skin. Abe, R., et al., (2004) J. Invest. Dermatol. 122, 461-467. HMGB1 is also up-regulated in malignant melanoma cells. Poser, I., et al., (2003) Mol. Cell. Biol. 23, 2991-2998. In biliary cancer, RAGE expression was associated with invasive potential in three cell lines in vitro. Hirata, K., et al., (2003) Hepatogastroenterology 50, 1205-1207. In gastric cancer, RAGE immunoreactivity correlated with increased lymph node metastasis, and HMGB1 was also observed to be increased. Kuniyasu, H., et al., (2002) J. Pathol. 196, 163-170. The same study also found that RAGE expression correlated with the invasiveness of gastric cancer cells in vitro and anti-sense oligomers against RAGE inhibited cell invasion. Kuniyasu, H., et al., (2002) J. Pathol. 196, 163-170. AGEs were shown to increase the proliferation of renal cell carcinoma cells. Miki, S., et al., (1993) Biochem. Biophys. Res. Commun. 196, 984-989. HMGB1 has been found to be associated with gastrointestinal stromal tumors, hepatocellular carcinoma, and osteosarcoma. Choi, Y. R., et al., (2003) Cancer Res. 63, 2188-2193; Kawahara, N., et al., (1996) Cancer Res. 56, 5330-5333; Charoonpatrapong, K., et al., (2006) J. Cell Physiol 207, 480-490. Taken together, these data support the assertion that RAGE and RAGE ligands are involved in nearly all malignancies.

Activation of RAGE has been shown to influence cell proliferation, survival, migration, and invasion in vitro and metastasis in vivo. Taguchi, A., et al., (2000) Nature 405, 354-360; Arumugam, T., et al., (2004) J. Biol. Chem. 279, 5059-5065; Arumugam, T., et al., (2005) Clin. Cancer Res. 11, 5356-5364; Kuniyasu, H., et al., (2002) J. Pathol. 196, 163-170; Huttunen, H. J., et al., (2002) Cancer Res. 62, 4805-4811. These effects are likely the result of RAGE activating the cellular signaling pathways that regulate these functions. RAGE is known to stimulate multiple signaling pathways crucial for cell proliferation, including MAP kinase (Erk1/2). Taguchi, A., et al., (2000) Nature 405, 354-360; Arumugam, T., et al., (2004) J. Biol. Chem. 279, 5059-5065. RAGE also activates signaling pathways thought to regulate cell migration, such as the Ras-extracellular signal-regulated kinase, Cdc42/Rac, stress-activated protein kinase/c-Jun-NH₂-terminal kinase, and p38 mitogen-activated protein kinase pathways. Lander, H. M., (1997) J. Biol. Chem. 272, 17810-17814; Huttunen, H. J., et al., (1999) J. Biol. Chem. 274, 19919-19924; Taguchi, A., et al., (2000) Nature 405, 354-360. Stimulation of RAGE also leads to the activation of the transcription factor NFκB. Arumugam, T., et al., (2004) J. Biol. Chem. 279, 5059-5065; Bierhaus, A., et al., (2001) Diabetes 50, 2792-2808. This pathway explains some of the effects of RAGE activation on cell survival, as a number of anti-apoptotic genes, including IAPs, Bel-XL, and Bel-2, are also influenced by activation of NFκB. Wu, J. T., et al., (2005) J. Surg. Res. 123, 158-169. Furthermore, several transcriptional targets of RAGE signaling, such as vascular cell adhesion molecule 1 and tissue factor, likely contribute to the interaction between tumor cells and vascular endothelium that may be involved in stimulating metastasis. Schmidt, A. M., et al., (1995) J. Clin. Invest 96, 1395-1403; Bierhaus, A., et al., (1997) Circulation 96, 2262-2271. Although activation of various intracellular signaling pathways can be seen in response to stimulation with different ligands, no adaptor protein for the transduction of intracellular signals by RAGE has been identified. One study has suggested a direct interaction between the cytoplasmic domain of RAGE and MAP kinase (Erk1/2). Ishihara, K., et al., (2003) FEBS Lett. 550, 107-113.

Cancer cells depend upon interactions with the cells within the cancer microenvironment. Gupta, G. P., et al., (2006) Cell 127, 679-695. The cells that reside within the tumor microenvironment or are recruited to this environment are affected by and, in turn, affect cancer cells. Important cells in the tumor microenvironment include those that compose the microvasculature, including endothelial cells and pericytes; those that produce the abundant extracellular matrix that makes up the bulk of the stroma, including fibroblasts and myofibroblasts; and cells of the immune system, including a variety of leukocytes such as macrophages. Most of these cells are known to express RAGE; therefore, RAGE ligands generated by cancer cells are likely to influence the tumor microenvironment. Likewise, as shown in FIG. 2, cells of the tumor microenvironment also produce RAGE ligands that can interact with RAGE on cancer cells. While there are obviously many other factors involved in the “crosstalk” between the microenvironment and cancer cells, RAGE and RAGE ligands play a significant role.

One of the ways in which RAGE may affect cancer, beyond its effects on cancer cells themselves, is through its ability to influence angiogenesis. Tumor growth depends upon the ability of the cancer cells to receive adequate oxygenation and nutrients. Folkman, J. (2006) Annual Review of Medicine 57, 1-18. Tumors develop a blood supply both by commandeering local vessels and by developing new vessels. The development of new vessels, involves the proliferation and migration of endothelial cells as well as pericytes. A variety of RAGE ligands have been shown to influence endothelial cells, including AGEs and HMGB1. Goldin, A., et al., (2006) Circulation 114, 597-605. It was reported that the inhibition of HMGB1 expression in colon cancer inhibited angiogenesis. van Beijnum, J. R., (2006) Blood 108, 2339-2348. Activation of RAGE increases endothelial cell number and induces expression of vascular endothelial growth factor (VEGF), a potent angiogenic factor. Yamagishi, S., et al., (1997) J. Biol. Chem. 272, 8723-8730. RAGE ligands have been found to induce other angiogenic factors, such as IL-8, through activation of NFκB. Treutiger, C. J., et al., (2003) Journal of Internal Medicine 254, 375-385. Activation of RAGE also influences the vasculature by increasing endothelial permeability to macromolecules, which is a condition commonly observed in tumors. Wautier, J. L., et al., (1996) Journal of Clinical Investigation 97, 238-243; Fukumura, D., et al., (2006) J. Cell Biochem.

RAGE and RAGE ligands in cancer may mediate their effects on fibroblasts. The role of fibroblasts in cancer is currently the subject of ongoing study; the differences between normal stroma and reactive tumor stroma being the subject of considerable interest. One primary role of fibroblasts is the elaboration of the prominent extracellular matrix composing the tumor stroma. Notwithstanding, fibroblasts play other important roles in cancer. Kalluri, R., et al., (2006) Nature Reviews Cancer 6, 392-401.

Fibroblasts are associated with cancer cells during cancer development and progression. The structural and functional contributions of fibroblasts are significant as there are important differences between fibroblasts in healthy tissues and those found in tumors. Fibroblasts produce growth factors, chemokines and extracellular matrix molecules that facilitate the angiogenic recruitment of endothelial cells and pericytes. In particular, the fibroblasts found in tumors are called “activated fibroblasts,” also sometimes referred to as myofibroblasts. RAGE appears to regulate fibroblasts. Studies show that skin fibroblasts respond to AGEs by increasing their expression of RAGE and the cytokine TNFα. Lohwasser, C., et al., (2006) Journal of Investigative Dermatology 126, 291-299. Activation of RAGE on synovial fibroblasts has been found to increase MCP-1 synthesis, which was sufficient to induce the chemotaxis of monocytes. Hou, F. F., et al., (2002) Journal of the American Society of Nephrology 13. RAGE activation was also found to lead to myofibroblast transdifferentiation of mesothelial cells in the kidney. De Vriese, A. S., et al., (2006) Nephrology Dialysis Transplantation 21, 2549-2555. RAGE may also influence fibroblasts through the up-regulation of important fibroblast growth factors, such as connective tissue growth factor. Twigg, S. M., (2001) Endocrinology 142, 1760-1769.

Other cellular targets of RAGE activity important in cancer include macrophages. Macrophages and their precursors, monocytes, respond to RAGE ligands, and macrophages are also producers of RAGE ligands. Hofmann, M. A., et al., (1999) Cell 97, 889-901; Hasegawa, T., (2003) Atherosclerosis 171, 211-218. Macrophages can act differently depending upon the circumstances. A clear distinction needs to be made between normal macrophages and tumor-associated macrophages (TAMs). Macrophages derived from healthy or inflamed tissues appear to act primarily in an anticancer manner, as they can directly lyse tumor cells and also induce an immune response against cancer cells.

The immune modulatory effects of macrophages include their ability to present tumor-associated antigens to T cells as well as express immunostimulatory cytokines that increase the proliferation and anti-tumor functions of T cells and natural killer cells. TAMs show greatly reduced levels of these activities and rather appear to facilitate angiogenesis and influence the invasiveness of cancer by stimulating extracellular matrix breakdown and remodeling as well as by increasing tumor cell motility and the egress of tumor cells in the blood vessels. Condeelis, J., et al., (2006) Cell 124, 263-266.

Activation of RAGE has been suggested to lead to the destruction of macrophages. Kuniyasu, H., et al., (2005) Am. J. Pathol. 166, 751-760. On the other hand, RAGE activity has been reported to increase the conversion of monocytes to macrophages and to stimulate macrophage function associated with inflammation and diabetes. Hofmann, M. A., et al., (1999) Cell 97, 889-901. Another way in which RAGE may influence macrophage function is through its ability to influence leukocyte adhesion and monocyte transendothelial migration. Rouhiainen, A., et al., (2004) Blood 104, 1174-1182. In fact, RAGE itself can act as a counter receptor for the leukocyte integrin Mac-1. Chavakis, T., et al., (2003) J. Exp. Med. 198, 1507-1515.

The following examples are provided to more fully illustrate some of the embodiments of the present invention. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

### Example I

### S100P Stimulates Pancreatic Tumor Growth In Vivo

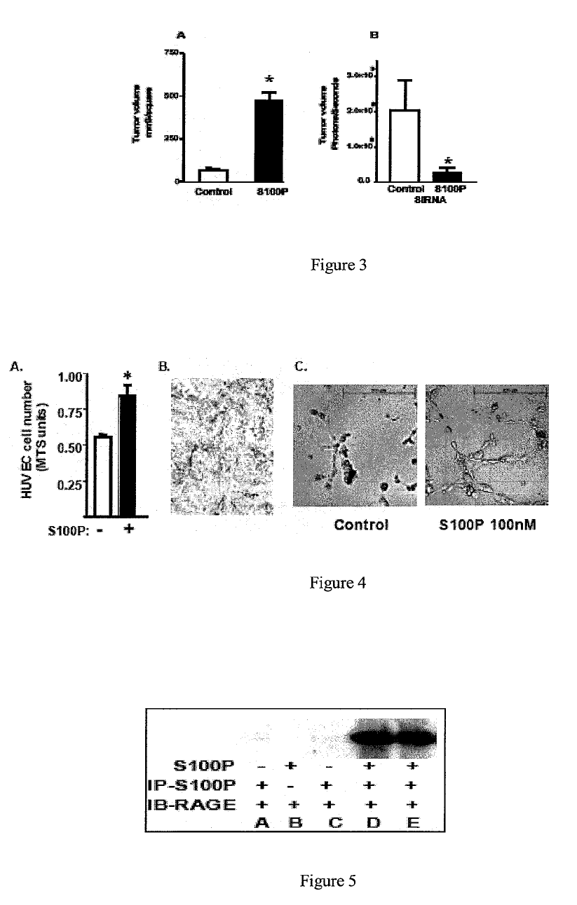

To directly determine the importance of S100P on pancreatic cancer, we examined the tumors formed from pancreatic cancer cell models over- and under-expressing S100P. We observed that WT Panc-1 cells, which do not express S100P, formed tumors, indicating that S100P is not required for tumor growth. However, expression of S100P in Panc-1 cells greatly increased the size of tumors as shown in FIG. 3A. Likewise, silencing of S100P by stable expression of a shRNA against S100P in BxPC3 cells reduced the size of tumors as shown in FIG. 3B. Similarly, we have observed that silencing S100P in Mpanc96 and MiaPaca2 cells also inhibits tumor growth in vivo (data not shown). Thus, while not necessary for tumor development, S100P stimulates the growth and the aggressiveness of pancreatic cancer. In FIG. 3A, athymic mice were inoculated subcutaneously with 1×10⁶ of either vector transfected or S100P expressing Panc-1 cells. Tumor volume was calculated after four weeks. In FIG. 3B, BxPC3 cells were stably transfected with control siRNA or S100P shRNA and also expressing the luciferase gene were transplanted orthotopically into scid mice. Bioluminescent imaging was utilized to estimate tumor volume from six animals in each group after six weeks. Data shown are mean±SE (*p<0.05).

### Example II

### S100P Stimulates Endothelial Cell Proliferation and Migration In Vitro

S100P is secreted by pancreatic cancer cells and acts in an autocrine manner to stimulate their growth, survival, and invasiveness. Thus, S100P could also interact with receptors on cells within the microenvironment. The effects of S100P on human umbilical vein endothelial cells (HUVECs) in vitro were examined. As shown in FIG. 4A, S100P treatment for 48 hrs caused a significant increase in HUVEC cell numbers. HUVEC cells were cultured in the presence of either S100P or uninduced bacterial protein for 48 hours and the number of cells was estimated using MTS. FIG. 4B shows CD31 staining of tumors formed from pancreatic cancer cells orthotopically implanted in nude mice. Tumors formed with BxPC3 cells were harvested and frozen sections were stained for CD31 staining with the help of the Cancer Biology Histology core. Microvessels bearing CD31 are stained brown. In a different assay, shown in FIG. 4C, Applicant observed that S100P increased endothelial cell migration and alignment when the cells were cultured on Matrigel. HUVEC cells were plated on Matrigel and followed for 4 hours. After this time, control cells mostly remained individually isolated while a few cells began to migrate and align themselves and occasional sprouting was observed. At 4 hrs, cells treated with S100P had mostly migrated aligned themselves, sprouted, formed closed polygons and a complex mesh like structure had developed.

### Example III

### S100P and RAGE

Whether S100P could interact with RAGE was investigated and found that it could in both pancreatic cancer and NIH 3T3 cells. As a direct indication of this interaction, lysates from Panel, and MiaPaca2 cells were incubated with S100P and S100P was then immunoprecipitated. The resulting product was washed, run on an SDS PAGE gel, and blotted with an antibody specific for RAGE as shown in FIG. 5. RAGE was not present in the samples run without addition of S100P or S100P antibody (lanes A-C), but RAGE was present when both these reagents were included (lanes D-F). These data indicate that S100P is able to interact directly with RAGE. In these experiments MiaPaca2 (A-D) and BxPC3-1 (E) cell lysates were incubated in the presence of S100P (100 ng) for 16 hours at 4° C. before immunoprecipitation with anti-S100P monoclonal antibodies (IP-S100P) (+) or control IgG (−) and RAGE was identified in the immunoprecipitates by western blotting with an anti-RAGE antibody (IB-RAGE).

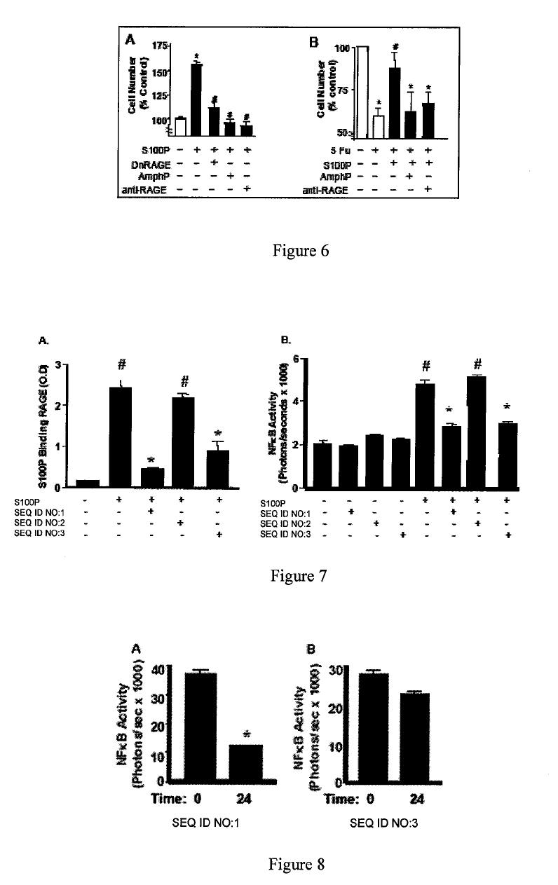

Based on the interaction between S100P and RAGE, it was hypothesized that the extracellular effects of S100P were mediated by RAGE. To test this hypothesis, the S100P/RAGE interaction was blocked in vitro using several different approaches including: 1) Expression of a dominant negative RAGE; 2) Use of a blocking antibody (anti-RAGE); or, 3) Use of an antagonistic peptide derived from amphoterin. Each of these treatments inhibited the effects of S100P on cell growth (FIG. 6A) and survival (FIG. 6B). FIGS. 6A and 6B show the effects of exogenous S100P are RAGE dependent. Wild-type NIH3T3 cells or cells expressing dominant negative RAGE (DnRAGE) were treated with 100 nM S100P(+) or with non-induced bacterial protein (−) and with (+) or without (−) a peptide antagonist (AmphP) or anti-RAGE antibodies (anti-RAGE). In FIG. 6A cell proliferation was determined on cells plated at equal numbers and treated for 48 hours. In FIG. 6B cell survival was determined in cells treated with or without 5-FU (150 ug/ml). Cell numbers were estimated by MTS assay. Data are % control and are means±SE for n=3 experiments. (*p<0.05 vs control; #p<0.05 vs 5-FU alone). Similar results were obtained with pancreatic cancer cell lines (data not shown) and indicate that the effects of exogenous S100P are mediated RAGE. Additionally, similar results have recently been obtained using RNAi approaches to silencing RAGE in vitro (data not shown).

### Example IV

### Peptides Based on the Structure of S100P Block S100P from Binding to Rage

A peptide antagonist of RAGE was previously developed from a COOH-terminal motif in HMGB1 (amphoterin) responsible for binding amphoterin with RAGE and also found to bind to ligand S100P. Huttunen, H. J., et al., (2002) Cancer Res. 62, 4805-4811.

Treatment with this peptide has shown inhibition of process extension and transendothelial migration of tumor cells. Furthermore, in an in vivo model of melanoma, the peptide significantly suppressed the formation of lung metastases. Arumugam, T., et al., (2004) J. Biol. Chem. 279, 5059-5065.

Using computer analysis, the structure similarities of amphoterin and S100P were compared. The region from position 32 to 63 of S100P corresponds to the sequence in amphoterin, believed to act as an antagonist of RAGE activation. To test the hypothesis that this region might be important for S100P interaction with RAGE, peptides were prepared corresponding to the first 10 amino acids referred to as SEQ ID NO: 1 (ELKVLMEKEL), the next 14 amino acids SEQ ID NO: 2 (KELPGFLQSGKDKD), and the last 13 amino acids SEQ ID NO: 3 (GKDKDAVDKLLKD).

Preliminary studies indicated that peptides of SEQ ID NO: 1 and SEQ ID NO: 3, but not SEQ ID NO: 2, could block the interaction of S100P with RAGE as indicated by a sensitive ELISA-type assay shown in FIG. 7A. The binding of purified S100P to RAGE was evaluated using an ELISA technique. Purified RAGE was coated into culture wells, S100P was allowed to bind in the presence and absence of the peptides (50 μM), the dishes were extensively washed, and bound S100P was detected with a specific antibody coupled to HRP.

Peptides SEQ ID NO: 1 and SEQ ID NO: 3, but not SEQ ID NO: 2, could also block the activation of NFκB by S100P in pant-1 cancer cells as shown in FIG. 7B. The effect of these novel peptide antagonists was evaluated on NFκB activity stimulated by S100P in Panc-1 cells. Panc-1 cells stably expressing the NFκB luciferase reporter gene were treated with or without S100P (100 nM) and either peptides of SEQ ID NOs: 1, 2 or 3 (1 μg/ml) for 4 hours. NFκB activity was estimated using luciferase quantitation. Numbers represent means±SE of triplicate determinations. * vs S100P alone (p<0.05); # vs control (p<0.05). Furthermore, administration of peptide of SEQ ID NO: 1 but not SEQ ID NO: 3 inhibited basal NFκB activity in S100P expressing BxPC3 cell tumors when administered in vivo as shown in FIG. 8. BxPC3 cells stably expressing an NFκB luciferase reporter were transplanted to scid mice and tumors were allowed to form. Animals were then injected i.p. with either peptide of SEQ ID NO: 1 (FIG. 8A) or SEQ ID NO: 3 (FIG. 8B). Imaging of luciferase activity was performed before and 24 h after peptide delivery. SEQ ID NO: 1 caused a significant reduction in NFκB activity whereas peptide SEQ ID NO: 3 did not. Numbers represent means±SE of triplicate determinations. *=p<0.05 vs time 0.

### Example V

### Peptide Antagonists Block Binding of Amphoterin to RAGE

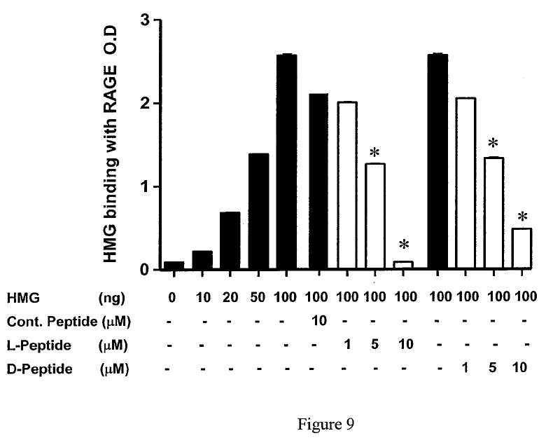

S100P peptide antagonists having the sequence Ac-ELKVLMEKEL-NH2 were made with standard L isomers of amino acids. This peptide antagonist comprises the amino acid sequence referred to as SEQ ID NO: 1 (ELKVLMEKEL), and in this instance the termini were blocked to prevent and/or reduce degradation. The inverse sequence, Ac-LEKEMLVKLE-NH2 was produced using D isomers of amino acids. This peptide antagonist comprises the amino acid sequence referred to as SEQ ID NO: 4 (LEKEMLVKLE), and in this instance the termini were blocked to prevent and/or reduce degradation. D isomers of amino acids, which are not naturally occurring, are thought to provide prolonged half-life in vivo. Both the peptide antagonist comprising SEQ ID NO:1 with blocked termini (also referred to herein for convenience as the “L-peptide”), and the peptide antagonist comprising SEQ ID NO:4 with blocked termini (also referred to herein for convenience as the “D-peptide”) were shown to compete for amphoterin (HMGB1) binding with RAGE. For this study, the extracellular domain of RAGE peptide (sRAGE) was coated onto the ELISA plate for 1 hour and non-specific binding sites were blocked with 1% BSA for 15 minutes. Amphoterin (0-100 ng) was added and incubated for 1 hour to allow its binding to its receptor and unbound molecules were removed by washing. Primary antibody against amphoterin and secondary antibody labeled with HRP and subsequently substrate for HRP was used to detect bound amphoterin with its receptor sRAGE. As shown in FIG. 9, the S100P peptide antagonists (L-peptide and D-peptide) competed with amphoterin and blocked its ability to bind with RAGE. A control peptide of the same size had no effect. *=p<0.05 versus HMGB1 alone.

### Example VI

### Peptide Antagonists Block S100P and S100A4 Binding to RAGE

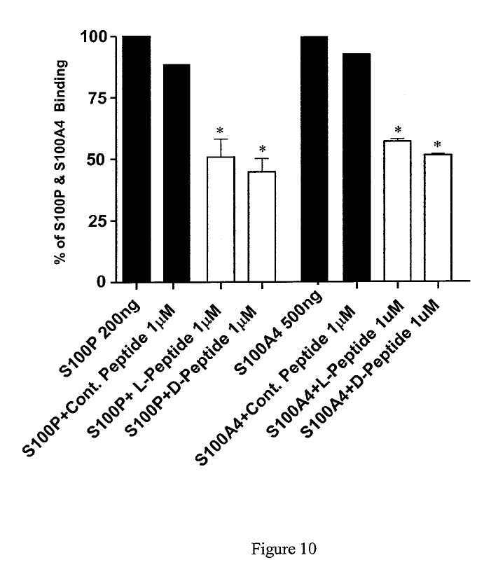

The assay of Example V was repeated with another two RAGE ligands S100P and S100A4, and their binding with RAGE was also blocked by S100P peptide antagonists (L and D). Thus, as can be seen from FIG. 10, S100P peptide antagonists (L-peptide and D-peptide) compete for binding of S100P and also S100A4 binding with RAGE. Thus, as described in this Example VI and the proceeding Example V, the L-peptide and D-peptide peptide antagonists which comprise SEQ ID NO: 1 or SEQ ID NO: 4, respectively, inhibit the interactions of amphoterin (HMGB1 or HMG-1), S100P, and S100A4 with RAGE. Accordingly, the L-peptide and D-peptide peptide antagonists should inhibit the binding and signaling effects of all RAGE agonists.

### Example VII

### Peptide Antagonists Inhibit S100P Activation of Rage as Indicated by Inhibition of Cellular NFκB Signaling

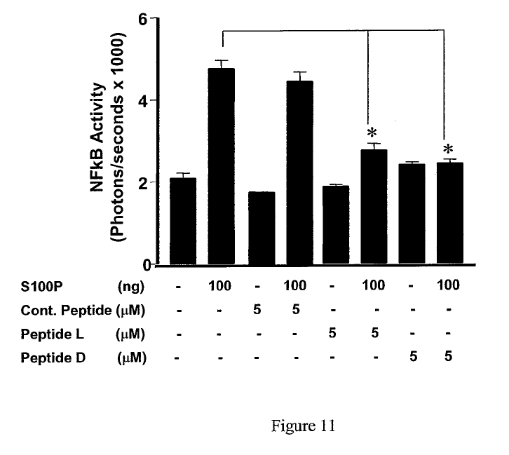

S100P has been shown to activate the intracellular transcription factor NFκB through its interactions with RAGE. In this assay, S100P peptide antagonists (L-peptide and D-peptide) were found to block S100P induced NFκB activity in pancreatic cancer MPanc-96 cells. MPanc-96 pancreatic cancer cells were stably transfected with a reporter plasmid, NFκB-Luc reporter, that provides a quantifiable photon output based on NFκB transcriptional activity. Extracellular addition of S100P to the cells induced the NFκB activity, which has previously been proven to be mediated through interaction with RAGE. Pre-treatment of the cells with S100P peptide antagonists (L-peptide and D-peptide), block S100P binding with its receptor RAGE, thus blocking S100P induced NFκB activity. Results of this assay are depicted in FIG. 11. The data indicate that the RAGE antagonism is functionally relevant. The results show that inhibition of S100P binding to RAGE not only occurs in the binding assays described in Example V and Example VI, but also in a functional assay based on the ability of S100P to activate the transcription factor NFκB through RAGE.

### Example VIII

### Peptide Antagonists Block Rage Function In Vivo as Indicated by Inhibition of Rage Activity after Intratumoral Injection in Subcutaneous Tumor

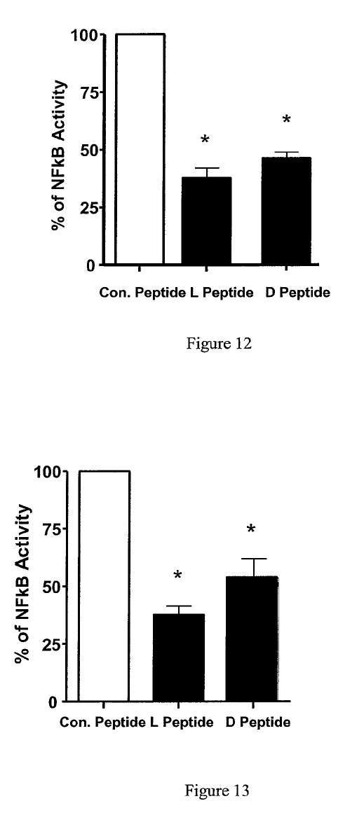

S100P peptide antagonists (L-peptide and D-peptide) block NFκB activity in vivo in subcutaneous tumors. MPanc-96 pancreatic cancer cells stably transfected with the NFκB-Luc reporter were injected into nude mice subcutaneously (1×10⁶) and in vivo NFκB activity indicated by luciferase was measured using an IVIS-100 bioluminescence system. S100P peptide antagonists (L-peptide and D-peptide) (10 mg/kg·b·wt) were injected intratumorally and NFκB activity was measured after 4 hours. As can be seen from FIG. 12, both peptides caused a strong reduction in NFκB activity, indicating their abilities to inhibit RAGE when delivered in vivo. Thus, delivery of peptide antagonists by direct injection in subcutaneous tumors inhibits NFκB as an indirect read-out of RAGE.

### Example IX

### Peptide Antagonists Block RAGE Function In Vivo as Indicated by Inhibition of RAGE Activity after Intraperitoneal Injection in Orthotopic Tumor

MPanc-96 pancreatic cancer cells stably transfected with the NFκB-Luc reporter were injected orthotopically into the pancreas (1×10⁶), and NFκB activity indicated by luciferase activity was measured after two weeks. S100P peptide antagonists (L-peptide and D-peptide) (10 mg/kg·b·wt) were injected intraperitoneally and NFκB activity in the orthotopic tumor was measured after 4 hrs. As shown in FIG. 13, both peptides caused a significant inhibition of cancer cell NFκB activity (p<0.05). Thus, delivery of antagonist peptides systemically by intraperitoneal injection still significantly inhibits NFκB as an indirect read-out of RAGE.

### Example X

### Variants of RAGE Antagonist Properties

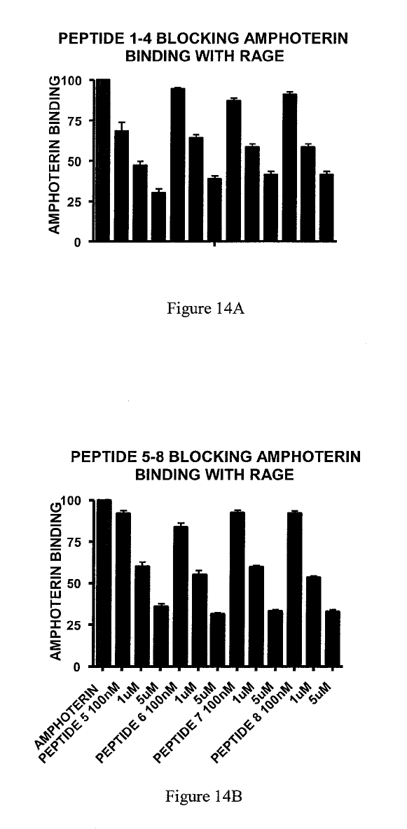

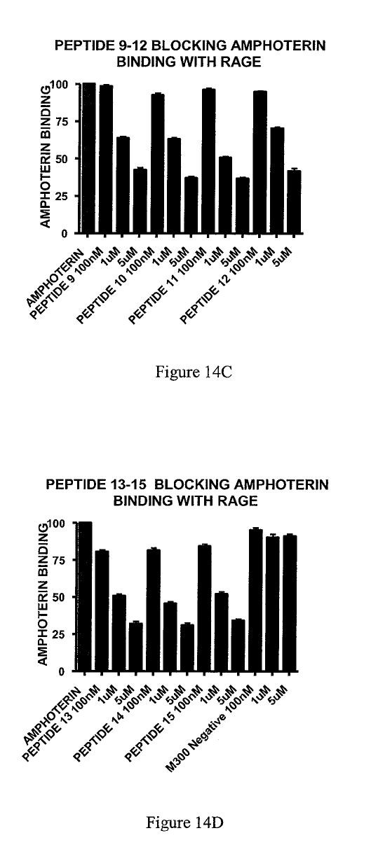

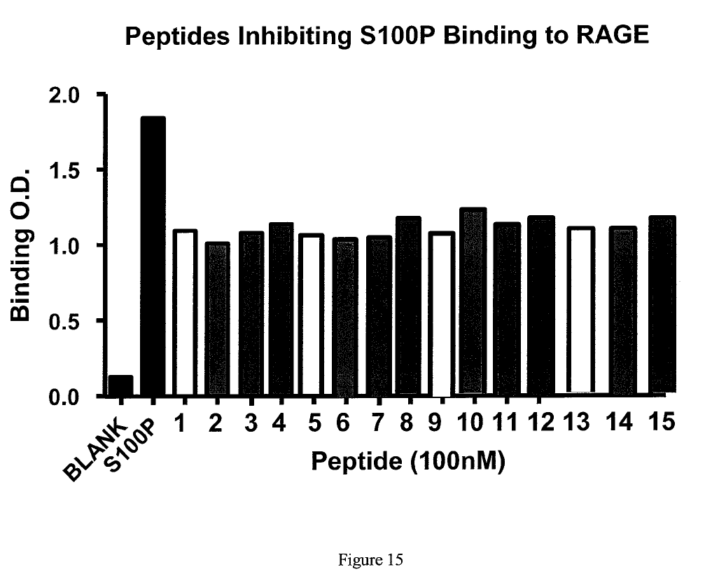

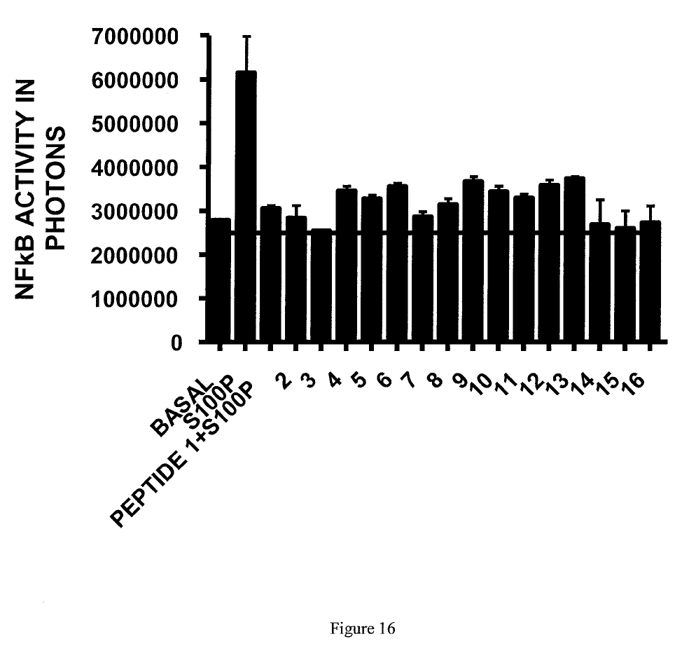

Experiments were performed to determine the sequence requirements of the RAGE antagonist properties of variants of SEQ ID NO:1. The peptides listed in Table 3 were used to compete with either HMGB1 (Amphoterin) (FIG. 14) or S100P (FIG. 15) for binding to RAGE in an in vitro assay. To further indicate whether changing the amino acid composition altered the ability of the peptide to inhibit RAGE we used a biological assay based on NFκB activation (FIG. 16). What these data indicate is that substitution of alanine for any individual amino acid in SEQ ID NO:1 did not make any significant difference in its ability to block agonist binding and activation of RAGE. Furthermore, two small peptides based on the sequence of amphoterin also inhibited RAGE binding. Thus, the ability of these peptides to inhibit RAGE activation is likely based on properties of the peptides such as charge distribution rather than specific amino acid interactions.

TABLE 3

| SEQ ID NO | Peptide # | Sequence | Amount |
|---|---|---|---|
| SEQ ID NO: 1 | 1. | ELKVLMEKEL | 1 mg |
| SEQ ID NO: 7 | 2. | LKVLMEKEL | 1 mg |
| Ac-SEQ ID NO: 1-NH₂ | 3. | Ac-ELKVLMEKEL- | 1 mg |
| | | NH₂ | |
| SEQ ID NO: 8 | 4. | ALKVLMEKEL | 1 mg |
| SEQ ID NO: 9 | 5. | EAKVLMEKEL | 1 mg |
| SEQ ID NO: 10 | 6. | ELAVLMEKEL | 1 mg |
| SEQ ID NO: 11 | 7. | ELKALMEKEL | 1 mg |
| SEQ ID NO: 12 | 8. | EAKVAMEKEL | 1 mg |
| SEQ ID NO: 13 | 9. | EAKVLAEKEL | 1 mg |
| SEQ ID NO: 14 | 10. | EAKVLMAKEL | 1 mg |
| SEQ ID NO: 15 | 11. | EAKVLMEAEL | 1 mg |
| SEQ ID NO: 16 | 12. | ELKVLMEKAL | 1 mg |
| SEQ ID NO: 17 | 13. | ELKVLMEKEA | 1 mg |
| SEQ ID NO: 18 | 14. | KLKEKYEKDI | 1 mg |
| SEQ ID NO: 19 | 15. | LKEKYEKDI | 1 mg |

Inhibition of the binding of amphoterin to RAGE by antagonist peptides was tested using an in vitro ELISA based binding assay. For this study, the extracellular domain of RAGE peptide (sRAGE) was coated onto the ELISA plate for 1 hour and non-specific binding sites were blocked with 1% BSA for 15 minutes. The plates were then treated with peptides 1-15 as defined in Table 3 at the indicated concentrations. Amphoterin (100 ng) was added and incubated for 1 hour to allow its binding to RAGE and unbound molecules were removed by washing. Primary antibody against amphoterin and secondary antibody labeled with HRP and subsequently substrate for HRP was used to detect bound amphoterin with its receptor sRAGE. As shown in FIG. 14, all of the peptides inhibited amphoterin binding to RAGE to a very similar extent.

Similarly, inhibition of the binding of S100P to RAGE by antagonist peptides was tested using an in vitro ELISA based binding assay. For this study, the extracellular domain of RAGE peptide (sRAGE) was coated onto the ELISA plate for 1 hour and non-specific binding sites were blocked with 1% BSA for 15 minutes. The plates were then treated with peptides 1-15 as defined in Table 3 at the indicated concentrations: S100P (100 ng) was added and incubated for 1 hour to allow its binding to RAGE and unbound molecules were removed by washing. Primary antibody against S100P and secondary antibody labeled with HRP and subsequently substrate for HRP was used to detect bound S100P with RAGE. As shown in this figure, all of the peptides inhibited S100P binding to RAGE to a very similar extent.

Further, inhibition of S100P activation of RAGE by antagonist peptides analyzed on pancreatic cancer cells in vitro. For this study, BxPC3 pancreatic cancer cells were stably transfected with a reporter for NFkB activity, NFkB-luc. Cells were then treated with peptides 1-15 as defined in Table 3 at a concentration of 100 uM. S100P (100 ng) was added and the effects on NFkB activity as measured by luciferase generated photon production was analyzed. Each of the peptides inhibited S100P stimulated RAGE activation of NFkB with a similar effectiveness.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It will be understood that certain of the above-described structures, functions, and operations of the above-described embodiments are not necessary to practice the present invention and are included in the description simply for completeness of an exemplary embodiment or embodiments. It is therefore to be understood that the invention may be practiced otherwise than as specifically described without actually departing from the spirit and scope of the present invention as defined by the appended claims and contemplated that the claims will cover any such modifications or embodiments that fall within the true scope of the invention.

We claim:

1. An isolated peptide comprising an amino acid sequence of SEQ ID NO: 1.

2. An isolated peptide comprising an amino acid sequence of SEQ ID NO: 2.

3. An isolated peptide comprising an amino acid sequence of SEQ ID NO: 3.

4. An isolated peptide comprising an amino acid sequence of SEQ ID NO: 4.

5. An isolated peptide comprising an amino acid sequence of SEQ ID NO: 5.

6. A pharmaceutical composition comprising a therapeutically effective amount of peptides of claim 1, 2, 3, 4, or 5 and an pharmaceutically acceptable carrier.

7. A method of inhibiting RAGE comprising administering a therapeutically effective amount of a peptide having an amino acid sequence of SEQ ID NO: 1 to a subject in need thereof.

8. A method of inhibiting RAGE comprising administering a therapeutically effective amount of a peptide having an amino acid sequence of SEQ ID NO: 3 to a subject in need thereof.

9. A method of inhibiting RAGE comprising administering a therapeutically effective amount of a peptide having an amino acid sequence of SEQ ID NO: 4 to a subject in need thereof.

10. A method of inhibiting RAGE comprising administering a therapeutically effective amount of a peptide having an amino acid sequence of SEQ ID NO: 4 to a subject in need thereof.

11. A method of treating cancer comprising administering a therapeutically effective amount of a peptide having an amino acid sequence of SEQ ID NO: 1 to a subject in need thereof.

12. A method of treating cancer comprising administering a therapeutically effective amount of a peptide having an amino acid sequence of SEQ ID NO: 3 to a subject in need thereof.

13. A method of treating cancer comprising administering a therapeutically effective amount of a peptide having an amino acid sequence of SEQ ID NO: 4 to a subject in need thereof.

14. A method of inhibiting RAGE comprising administering a therapeutically effective amount of a peptide having an amino acid sequence of SEQ ID NO: 5 to a subject in need thereof.

15. A peptide as recited in claim 1, 3, 4, or 5 for use in the manufacture of a medicament for the prevention or treatment of a disease or condition ameliorated by the inhibition of RAGE.

16. A method of treatment of a RAGE-mediated disease comprising the administration of a therapeutically effective amount of a compound as recited in claim 1 and another therapeutic agent.

17. A method of treatment of a RAGE-mediated disease comprising the administration of a therapeutically effective amount of a compound as recited in claim 3 and another therapeutic agent.

18. A method of treatment of a RAGE-mediated disease comprising the administration of a therapeutically effective amount of a compound as recited in claim 4 and another therapeutic agent.

19. A method of treatment of a RAGE-mediated disease comprising the administration of a therapeutically effective amount of a compound as recited in claim 5 and another therapeutic agent.

20. An isolated nucleic acid comprising a sequence that encodes a peptide consisting of the amino acid sequence of SEQ ID NO: 1.

21. An isolated nucleic acid comprising a sequence at least 90% identical to SEQ ID NO: 6, wherein the nucleic acid encodes a polypeptide that binds to RAGE.

22. A pharmaceutical composition comprising a therapeutically effective amount of a nucleic acid of claim 20 and an pharmaceutically acceptable carrier.

23. A method of inhibiting RAGE comprising administering a therapeutically effective amount of a nucleic acid sequence comprising a nucleic acid sequence of SEQ ID NO: 6 to a subject in need thereof.


SEQUENCE LISTING (19 sequences)

SEQ ID NO: 1 - protein, 10 aa, Homo sapiens
ELKVLMEKEL

SEQ ID NO: 2 - protein, 14 aa, Homo sapiens
KELPGFLQSGKDKD

SEQ ID NO: 3 - protein, 13 aa, Homo sapiens
GKDGDAVDKLLKD

SEQ ID NO: 4 - protein, 10 aa, Homo sapiens
LEKEMLVKLE

SEQ ID NO: 5 - protein, 13 aa, Homo sapiens
DKLLKDVADGDKG

SEQ ID NO: 6 - DNA, 30 bp, Homo sapiens
gagctcaaggtgctgatggagaaggagcta

SEQ ID NO: 7 - protein, 9 aa, Homo sapiens
LKVLMEKEL

SEQ ID NO: 8 - protein, 10 aa, Homo sapiens
ALKVLMEKEL

SEQ ID NO: 9 - protein, 10 aa, Homo sapiens
EAKVLMEKEL

SEQ ID NO: 10 - protein, 10 aa, Homo sapiens
ELAVLMEKEL

SEQ ID NO: 11 - protein, 10 aa, Homo sapiens
ELKALMEKEL

SEQ ID NO: 12 - protein, 10 aa, Homo sapiens
EAKVAMEKEL

SEQ ID NO: 13 - protein, 10 aa, Homo sapiens
EAKVLAEKEL

SEQ ID NO: 14 - protein, 10 aa, Homo sapiens
EAKVLMAKEL

SEQ ID NO: 15 - protein, 10 aa, Homo sapiens
EAKVLMEAEL

SEQ ID NO: 16 - protein, 10 aa, Homo sapiens
ELKVLMEKAL

SEQ ID NO: 17 - protein, 10 aa, Homo sapiens
ELKVLMEKEA

SEQ ID NO: 18 - protein, 10 aa, Homo sapiens
KLKEKYEKDI

SEQ ID NO: 19 - protein, 9 aa, Homo sapiens
LKEKYEKDI